(19) United States
(12) Patent Application Publication
(10) Pub. No.: US 2018/0140537 A1
(43) Pub. Date: May 24, 2018

(54) ACTIVE COMPLEX FOR A COSMETIC PRODUCT AGAINST SKIN AGEING

(71) Applicant: Franck MARGNAT, Jr., Marseille (FR)

(72) Inventor: Franck MARGNAT, Jr., Marseille (FR)

(21) Appl. No.: 15/306,544

(22) PCT Filed: Nov. 27, 2015
(86) PCT No.: PCT/FR2015/053247

§ 371 Date: Oct. 25, 2016

(30) Foreign Application Priority Data
Dec. 8, 2014 (FR) 1462075

(51) Int. Cl. A61K 8/99; A61Q 19/08; A61K 8/9789; A61K 8/73; A61K 8/9794; A61K 8/02

(52) U.S. Cl. CPC A61K 8/99; A61Q 19/08; A61K 8/9789; A61K 8/0212; A61K 8/9794; A61K 8/0216; A61K 8/735

(57) ABSTRACT

An anti-age active complex and a cosmetic product for the skin having at least the following elements:
thalassin,
prickly pear oil, and
hyaluronic acid or salt thereof, and further preferably
white lily flower extract, and
spirulin.

The present invention relates to an active complex designated as an anti-age complex having a protective cutaneous effect, notably a regenerating or invigorating effect, intended to prevent or control skin ageing, notably wrinkles, said complex comprising an association of a plurality of active substances.

The present invention also relates to a cosmetic or dermatological composition comprising a said complex according to the invention and excipients for application via a topical route.

The skin comprises a surface layer formed by the epidermis, and deeper layers forming the dermis. The surface layer forming the epidermis mainly consists of keratinocytes (85 to 90% of the epidermal cells). The thicker dermis mainly consists of collagen, elastin and proteoglycans. These three types of molecules are synthesized by dermal fibroblasts. Skin ageing may be natural or caused by the environment, including weather aggressions, which may notably contribute to accelerating degradation of the collagen of the dermis, and in particular exposure to the UV rays from the sun, temperature variations and free radicals.

Many compositions having preventive effects or controlling skin ageing, are known, notably topical compositions based on suitable plant extracts preferentially stemming from plants known for their favorable properties in WO 02/060394 and WO 2009/050866.

However, there always exists a need to have available novel alternative topical compositions giving the possibility of efficiently controlling the effects of skin ageing.

Hyaluronic acid and its sodium salt are recognized for their moisturizing properties. Spirulin is a micro-algae which is very rich in proteins, iron, beta-carotene, vitamins, minerals, trace elements; it is known as a food supplement with antiseptic properties. Thalassin is a substance extracted from the plant Tripleurospermum Maritimum which contributes to relaxing the muscles of the face and smoothing out the features of the face. Prickly pear oil which is a very rare oil rich in vitamin E and essential fatty acids which have anti-oxidant and anti-radical properties. White lily extract is known for its healing and calming properties.

According to the present invention, it was sought to provide a novel complex of a mixture of active substances designated hereafter as a “active mixture”, having an anti-ageing skin effect, both at the epidermis with an anti-oxidant effect and at the dermis with stimulation of the production of collagen and a reduction of the enzymes which break down collagen, the dermis being considered as the main location of deleterious damages inflicted by ageing.

The “anti-age” potential of the mixtures of actives according to the present invention was evaluated on keratinocyte cells and on human fibroblasts exposed to ultraviolet radiations by evaluating their modulating effects towards photo-induced functional alterations of biological markers. The epidermal oxidation level was evaluated by measuring the oxidizing reactive species (known as “ROS”) on HaCaT keratinocytes as a marker of epidermal photo-ageing. The synthesis and the degradation of the extracellular matrix were evaluated by measuring the levels of procollagen I and MMP-1 on NHDF fibroblasts as markers of dermal photo-ageing.

An anti-age activity index designated as “AAC” was determined according to the present invention, which is a quantitative parameter of the anti-ageing skin potential of a test element taking into account a protective effect at the epidermis by integrating an epidermal anti-oxidant effect score and a protective effect at the dermis by integrating a dermo-protective effect score towards the synthesis of collagen, a main marker of the dermal extracellular matrix (ECM) and/or towards the degradation of metalloproteinase-1 (MMP-1) which is a collagenase enzyme produced by the fibroblasts of the dermis degrading the dermal extracellular matrix (ECM). Still more specifically, a capability is sought for opposing the reduction of collagen synthesis following UV stress and opposing the increase in the production of metalloproteinase-1 for ensuring dermal homeostasis.

More specifically, according to the present invention, an AAC potential is defined as follows:

AAC==AOE+½DPC+½ DPM with

[AOE]: % of epidermal anti-oxidant potential as defined hereafter.

[DPC.]: % of dermo-protective collagen potential as defined hereafter.

[DPM]: % of dermo-protective MMP1 potential as defined hereafter.

AOE=% of reduction of ROS after treating keratinocyte cells with UV in the presence of the complex according to the invention as compared with a control having undergone a same UV treatment in the absence of a test complex according to the present invention or other treatment.

DPC=% increase in collagen after treating NHDF fibroblast cells with UV in the presence of the complex according to the invention as compared with a control having undergone a same UV treatment in the absence of the test complex according to the present invention or other treatment.

DPM=% of reduction in metalloproteinases induced after treatment of NHDF fibroblasts with UV in the presence of the complex according to the invention as compared with a control having undergone a same UV treatment in the absence of the test complex according to the present invention or other one.

Another object of the present invention is to provide anti-age complexes having AAC indexes of at least 100, preferably at least 110 with cumulatively:

an AOE at least equal to 70, preferably at least 80, and

a DPC at least equal to 45, preferably at least equal to 50, and

a DPM at least equal to 15, preferably at least equal to 30.

Document DE 102009045981 describes the benefit of a cosmetic composition comprising at least 13 active substances (A to M) including an extract of Tripleurospermum and of hyaluronic acid. In spite of the very large number of additional components which may be included in the composition, no mention is made of a prickly pear seed oil extract. In document DE 102009045981, no serious documented result is provided relating to anti-age properties, whether this be in terms of epidermal anti-oxidant potential and/or thermo-protective potential.

Document US 2009/116211 describes cosmetic compositions containing a prickly pear seed oil extract. It is indicated that this substance promotes the synthesis of collagen and of hyaluronic acid at skin cells. However, in document US 2009/116211, no data are provided relating to the epidermal antioxidant AOE potential or dermo-protective potential MMP1 as defined above of the tested composition.

To do this, the present invention provides an anti-age active complex for skin comprising at least the following active elements:

thalassin, extracted from Tripleurospermum Maritimum,

prickly pear seed oil, and

hyaluronic acid or a salt thereof.

The comparative assays provided in the detailed exemplary embodiments hereafter gave the possibility of establishing that the anti-age potential of the combinations according to the present invention comprising at least the three active elements defined above are superior to all the tested binary combinations.

More particularly, the complex according to the invention in liquid form comprises:

0.2 to 3% by weight of an aqueous solution of thalassin with 2% by weight of thalassin,

0.02 to 2% by weight of prickly pear oil, and

0.02 to 0.2% by weight of hyaluronic acid or a salt thereof.

Preferably, in the comparative assays of the examples hereafter, it was discovered that the highest anti-age skin potential was obtained when the complex according to the invention further comprises at least the two following additional active elements:

white lily (Lilium candidum) flower extract, preferably a hydro-glycolic solution of white lily flower (Lilium candidum), and

spirulin, extracted from Spirula Maxima, preferably a spirulin hydro-glycolic solution.

More particularly, the complex according to the invention in liquid form comprises:

0.02 to 0.15% by weight of a hydro-glycolic solution containing 5% by weight of said white Lily extract,

0.002 to 0.05% by weight of a hydro-glycolic solution containing 1% by weight of spirulin.

Still more particularly, the complex according to the invention in liquid form comprises at least:

1 to 2% by weight of an aqueous solution of thalassin with 2% by weight of thalassin,

0.1 to 0.2% by weight of said prickly pear oil, and,

0.1 to 0.2% by weight of hyaluronic acid or a salt thereof.

Still more particularly, the complex according to the invention in liquid form further comprises at least:

0.01 to 0.1% by weight of a hydro-glycolic solution containing 5% by weight of said white lily extract, and

0.01 to 0.1% by weight of a hydro-glycolic solution containing 1% by weight of spirulin.

Still more particularly, the complex according to the invention in liquid form comprises:

1.6% by weight of said thalassin solution

0.16% by weight of said prickly pear oil

0.16% by weight of hyaluronic acid.

Still more particularly, in liquid form comprises:

1.6% by weight of said thalassin solution,

0.16% by weight of said prickly pear oil,

0.16% by weight of hyaluronic acid,

0.06% by weight of said hydro-glycolic solution of said white lily extract, and

0.03% by weight of said hydro-glycolic solution of spirulin.

The present invention also provides a cosmetic or dermatological product comprising a said complex according to the invention in combination with cosmetically or pharmaceutically acceptable supports and/or excipients.

Non-cytotoxic effective doses of the said different active elements of the complex as defined above will be applied.

Still more particularly, an anti-ageing cosmetic product according to the invention is formulated for topical application as a gel, lotion, cream, pomade, soap, paste for a mask.

The topical compositions according to the invention may comprise various customary excipients adapted for external topical administration, in particular dermatologically and cosmetologically acceptable excipients. These suitable excipients for the formulation are well known to one skilled in the art and for example comprise agents for promoting penetration, moisturizing agents, thickeners, emollients and surfactants, emulsifiers; preservatives.

These topical administration forms are prepared by known techniques, and for example, in the case of a cream, by dispersing a fatty phase in an aqueous phase in order to obtain an oil-in-water emulsion, or conversely for preparing a water-in-oil emulsion.

In a particular embodiment, the cosmetic product according to the invention is formulated as a cream comprising said elements of said complex and at least the following additional excipients: emulsifier, emollient, thickener, moisturizer and preservatives and a pH adjusting agent.

In a particular embodiment, the cosmetic product according to the invention is formulated as an aqueous gel comprising, in a homogenous mixture in water, said elements of said complex and at least the following additional excipients: gelling agent, thickener, moisturizer and preservative and pH adjusting agent.

The preparations generally contain other excipients and/or auxiliaries and sometimes other active ingredients, for example coloring agents, coloring pigments, radical scavengers, perfumes, stabilizers.

The cosmetic products may also comprise auxiliaries and sometimes other active ingredients, for example antioxidant vitamins such as vitamin E, vitamin C, antioxidant agents like natural polyphenols, enzymes, plant active ingredients, natural anti-inflammatory substances, alcohols, polyols, esters, electrolytes, polar and non-polar oils, polymers, copolymers, phospholipids, coloring agents; perfumes; or further skin exfoliants.

The object of the invention is further a cosmetic product for treating skin for controlling signs of skin ageing, and in particular expression wrinkles caused by uncontrolled facial muscle contractions, consisting of applying on areas of the skin requiring such a treatment, a topical cosmetic product according to the invention.

Other features and advantages of the present invention will become better apparent upon reading the description of the examples which will follow, made illustratively and in a non-limiting way.

### EXAMPLE 1

### Anti-Age Active Complexes

The cytotoxicity and the anti-age potential of several complexes (mixtures) of actives was evaluated hereafter on human skin cells being cultivated.

1) Various complex mixtures were tested from the following active elements.

1.1) Aqueous thalassin solution.

Thalassin is extracted from the plant Tripleurospermum Maritimum from the family of Asteraceae consisting of the following elements with the following contents:

Amino acids: Alanine (10.32%), Glycine (1.64%), Valine (7.14%), Leucine (4.82%), IsoLeucine (3.45%), Threonine (2.22%), Proline (23.18%), Asparagine (10.56%), Aspartic acid (12.63%), Phenylalanine (4.64%), Tyrosine (0.92%),

Fructo-oligosaccharides: fructans in an amount of 0.08% by weight,

Vitamins B5 (0.03×10⁻³%), B6 (33.510⁻⁶%), PP (0.27 10⁻³%), B9 (3.10⁻⁶%),

Sugars: fructose (90.5×10-3%), saccharose (32.4×10⁻³%),

Minerals: Ca (9.9×10⁻³%), Mg (4.8×10⁻³%), Na (9.8×10⁻³%), K (134.9×10⁻³%), Zn (0.05×10⁻³%) and Mn (0.02×10⁻³%).

This is an aqueous liquid extract marketed by BIOTECH MARINE (France Z. I. BP 72 2260 PONTRIEUX) under the reference of THALASSINE 2. Such an active contains the elements listed above with the contents listed of the Tripleurospermum Maritirnum extract above in a content of 2% by weight in solution in water (97.8%) and in sorbic acid (0.2% by weight).

1.2) White lily or Lilium candidum flower hydro-glycolic solution.

The liquid used is marketed by IES LABO France (04700 ORAISON) under reference EH354. This solution is obtained by filtration after long ripening and stirring of the plant in a mixture of water and propylene glycol at room temperature. After withdrawal of the plant, such a solution contains in % by weight:

5% of plant extract,

45% of propylene glycol,

45% of water.

The constituents extracted from Lilium candidum are lipophilic actives comprising an essential oil rich in aromatic compounds, phytosterols (spirostan, furostan, beta-sitosterol) and steroidal alkaloids (etiolin).

Its physico-chemical characteristics are:

aspect: brown to yellow liquid,

specific gravity at 20° C.: 1,050 to 1,070 g/cm³

pH=3.5-5.5;

refractive index at 20° C:1.68 to 1.410.

1.3) Spirulin hydro-glycolic solution.

Spirulin is extracted from a micro-alga which is found in lakes of the intertropical belt. It essentially consists of about 50-75% of plant proteins, mixed with vitamins A, E, B1, B2, B3, B6, B7, B8, K and beta-carotene, of minerals and trace elements such as Ca, P, Mg, Fe, Zn, Cu, Mn, Cr, Na, K, and Se. It is very rich in chlorophyll and phycocyanin. It also comprises enzymes including superoxide dismutase (SOD) which contains Fe. Finally, it contains essential fatty acids: omega 6, gamma-linolenic acid in a large amount.

The liquid lotion used is a spirulin hydroglycolic solution containing an extract of Spirulina Maxima marketed by GREENTECH.FRANCE 63360 SAINT-BEAUZIRE) under the reference 300656. Such a solution contains in weight %:

1% of dry extract of Spirulina Maxima

49.5% of water, and

49.5% of butylene glycol.

1.4) prickly pear seed oil.

The prickly pear cactus is a kind of cactus (Opuntia ficus-indica) which produces seeds from which an oil rich in vitamin F and in alpha-linolenic acid, in vitamin E and sterols is extracted.

The oil used is marketed by IES LABO France (04700 ORAISON) under reference HV141. This oil is obtained by cold pressing of the seeds of the plant from Tunisia. The oil contains:

10 to 14% of C16:0 palmitic acid;

1% of C16:1 palmitoleic acid;

2.5 to 5.5% of C18:0 stearic acid;

20 to 30% of C18:1 oleic acid;

55 to 65% of C18:2 linoleic acid,

1% of C18:3 linolenic acid.

1.5) Hyaluronic acid.

Hyaluronic acid is a glycosaminoglycan polymer of 10² to 10⁴ kDa consisting of disaccharide units of the D-glucuronic acid and D-N-acetylglucosamine type, the glycoside bonds being in alternation: beta 1-4 and beta 1-3.

Hyaluronic acid was used in powder form containing more than 91% by weight of sodium hyaluronate for example marketed by SHANDONG FOCUSCHEM BIOTECH CO. LTD, CHINA). It is produced by bacterial fermentation with the bacterium Streptococcus zooepidemicus.

2) CYTOTOXICITY.

For each of the substances above, the maximum non-cytotoxic doses were determined, corresponding to cell viability ≥90% (hereafter “NCD”). Cell viability was evaluated by a neutral red test (NR) on human keratinocyte cells (HaCaT line) and on fibroblasts described hereafter after contact for 48 h. The substances were poured into the culture medium DMEM+10% FCS.

RESULTS

| | NCD (%) |
|---|---|
| | by |
| Assay elements | weight |
| THALASSIN solution | 3% |
| LILY FLOWER HYDROGLYCOLIC solution | 0.15% |
| SPIRULIN HYDROGLYCOLIC solution | 0.05% |
| PRICKLY PEAR OIL | 2% |
| SODIUM HYALURONATE POWDER | 0.2% |

3) ANTI-AGE ACTIVITY.

The anti-age potential of several complexes (mixtures) of actives was evaluated hereafter on cultivated human skin cells. An experimental in vitro approach based on the production of ROS by human epidermal keratinocytes after exposure to UV-B was proposed for appreciating the antioxidant potential of the complexes of mixtures of actives. The dermo-protective effects of the complexes were appreciated by measuring collagen I and MMP-1 levels on human dermal fibroblasts subject to UVs.

The complexes of mixtures [CAAL-2], [CAAL-3] and [CAAL-4] were directly prepared in the culture medium, from the various aforementioned actives, according to the formulae of table I hereafter with concentrations in weight %.

TABLE I

| ANTI-AGE COMPLEX | CAAL-2 | CAAL-3 | CAAL-4 |
|---|---|---|---|
| THALASSIN | 1.6% | 1.6% | — |
| LILY HYDROGLYCOLIC | 0.06% | — | 0.06% |
| SOLUTION | | | |
| SPIRULIN HYDROGLYCOLIC | 0.03% | — | 0.03% |
| SOLUTION | | | |
| PRICKLY PEAR OIL | 0.16% | 0.16% | 0.16% |
| HYALURONIC ACID | 0.16% | 0.16% | — |

The study was carried out on human dermal fibroblasts [NHDF] and human keratinocytes (HaCaT).

The NHDF cells are human dermis fibroblasts in a primary culture which were isolated and then kept and cultivated in the laboratory. The cells [NHDF] isolated from the skin from the foreskin of young children are cultivated in a D-MEM (Gibco®) medium supplemented with fetal calf serum (10% FCS) and with antibiotics, according to techniques set up in the laboratory, at 37° C. in an air-CO2 (95%-5%) humid atmosphere.

The HaCaT cells are human keratinocytes from an immortalized cell line which is kept in the laboratory (Normal Keratinization in a Spontaneously Immortalized Aneuploid Human Keratinocyte Cell Line N. E Fusenig, J. Cell. Biology; 106, 1988). The HaCaT cells are cultivated, in a D-MEM (Gibco®) medium supplemented with fetal calf serum (10% FCS) and with antibiotics, at 37° C. in an air-CO₂ atmosphere (95%-5%).

The HaCaT and NHDF cells are cultivated in flask of 25 or 75 cm² and are regularly passed before attaining confluence.

3.1) ANTI-OXIDANT EPIDERMAL POTENTIAL (“AOE” hereafter).

3.1.1) The principle of the assay is based on the measurement of the intracellular oxidation level by means of the probe: 2′,7′-dichloro-dihydrofluorescein diacetate (DCFH-DA). Non-fluorescent DCFH-DA penetrates by passive diffusion into the cells. After cleavage of the acetate groups by the esterases, DCFH accumulates at the cytosol. The oxidation of DCFH by the reactive oxygen species (ROS: “Reactive Oxidative Species”) leads to the formation of a fluorescent compound. The DCF fluorescence intensity gives the possibility of evaluating the oxidation level of the cells subject to an oxidative stress.

More specifically, the measurement of the fluorescence intensities of DCF gives the possibility of evaluating the oxidation level of the cells subject to an oxidative stress. The DCFH assay detects a large range of ROSes: hydroxyl peroxyl and other ROSes (Fluorescence probes used for detection of reactive oxygen species, A. Gomes et al., J. Biochem. Biophys. Methods, 65:45-80; 2005).

HaCaT keratinocytes cultivated in a mono-layer are detached by trypsinization. The cell suspension is sown in 24-well plates.

2 plates are sown in parallel: 1 plate [DCFH] (measurement of DCFH fluorescence) and 1 plate [Prot] (measurement of cell proteins).

The plates [DCFH] and [Prot] are treated in parallel, in an identical way, according to the following experimental procedure.

72 hours after sowing, the culture medium is removed and replaced with fresh medium containing the assay element. The cells are replaced at 37° C. and incubated for 24 hours, in an air-CO₂ atmosphere (95%/5%). After incubation with the assay element, the cells are rinsed with HBSS (a so called Hanks solution) and then exposed to UV-B (Philips TL-K 40 W ACTINIC BL REFLECTOR tubes) through the HBSS. The cells were treated with the various assayed complexes before (24 h) and after (24 h) irradiation with UV-B for about 30 minutes at 20 mJ/cm².

After irradiation, the HBSS is replaced with fresh medium containing the assay element. The cells are replaced at 37° C. for 24 h in an air-CO₂ atmosphere (95%/5%). At the end of the assay, the [DCFH] and [Prot] plates are treated, in the following way:

Plate [DCFH]:

The [DCFH] plates are emptied and rinsed with PBS. The cells are then incubated at 37° C., in the presence of a DCFH-DA solution. After incorporating the probe, the cells are rinsed with PBS. An extraction buffer (Triton X-100 1%) is added into each well. After 45 mins of incubation, the lyzates are sampled, transferred into 96-well plates. The fluorescence intensities (λExc: 485 nm, λEm: 535 nm) are expressed in relative fluorescence units (RFU).

Plate [Prot]:

A dosage of the proteins (BCA protein assay) is carried out in parallel in order to correct the [DCFH] values (RFU/well) according to the cell protein level (pg/well) in order to do without a variation in the number of cells related to an effect of the assay element on cell growth.

3.1.2) Results

The assay was carried out on HaCaT keratinocytes (HaCaTp91 strain) cultivated in a [DMEM+1% FCS] medium. The intracellular oxidation level was measured after treatment and irradiation of the cells.

The 3 complexes of mixtures designated as CAAL-2, CAAL-3 and CAAL-4 were assayed comprising the concentrations of various actives indicated in table I above.

Vitamin E (Tocopherol, 1 mM) was assayed in parallel as a positive control.

The intracellular oxidation level (F.R.U/well: relative fluorescence unit) recorded at the control and treated cultures was corrected according to the average cell protein level (μg/well). The values [DCFH] are expressed in arbitrary units (from the optical densities): [DCFH]=(F.R.U/μg prot.).

The data are recorded in table II.

The results (average +/−standard deviation, n=3) are collected in the table hereafter.

The “stat.” values p obtained with Student's test t are less than or equal to 0.01.

TABLE II

| | | |
|---|---|---|
| DCFH ASSAY | UV-induced ROS | Stat. |

(−) UV

| | | |
|---|---|---|
| Control 1 | 12.80 +/− 0.99 | (%) |

(+) UV

| | | | | |
|---|---|---|---|---|
| Control 2 | 134.12 +/− 2.23 | 121.32 | 100% | |
| [CAAL-2] | 18.12 +/− 0.96 | 5.32 | 4% | p ≤ 0.01 |
| [CAAL-3] | 45.31 +/− 4.81 | 32.51 | 27% | p ≤ 0.01 |
| [CAAL-4] | 74.31 +/− 4.23 | 61.51 | 61% | p ≤ 0.01 |
| VE | 35.72 +/− 2.66 | 22.92 | 19% | p ≤ 0.01 |

The exposure of the control cells to UVB is expressed by a highly clear increase in the intracellular oxidation level. The basal level of the ROSes (non-irradiated cultures: control 1) is multiplied by 10.5 after UV exposure of the cells (control 2).

The results show that the treatment of the cells with [CAAL-2], [CAAL-3] and [CAAL-4] gives the possibility of very clearly reducing the ROSes induced by the UVs ([ROSUV]) relatively to a control (control 2) having been subject to the same UV treatment but in the absence of an assay complex according to the present invention or other. It should be noted that the epidermal anti-oxidant potential varies according to the relevant mixture as follows:

CAAL-2: AOE=96% reduction of [ROSUV]

CAAL-3: AOE=73% reduction of [ROSUV]

CAAL-4: AOE=39% reduction of [ROSUV]

AOE=reduction % of ROSes induced after treating keratinocyte cells with UVs in the presence of the complex according to the invention relatively to a control having been subject to a same UV treatment in the absence of the assay complex according to the present invention or other.

The differences recorded at the batches CAAL-2, CAAL-3 and CAAL-4 are statistically significant relatively to the control (+) UV (p≤3.01, Student's test t).

The maximum antioxidant effect is recorded with [CAAL-2] which almost completely opposes the oxidative stress induced by the UV.

Under the same experimental conditions, the treatment of the HaCaT cells with vitamin E (-tocopherol, 1 mM) gives the possibility of reducing by 81% the oxidative stress induced by the UV.

3.2) DERMO-PROTECTIVE POTENTIAL.

3.2.1) The principle of the assay is based on the evaluation of 2 parameters—neosynthesis of collagen I and production of MMP-1—on human fibroblasts [NHDF] cultivated in the absence or in the presence of the assay element, and stimulated by UV-A.

The level of procollagen I is quantified by assaying [PIP] (procollagen of the I peptide type) in incubation media by means of an ELISA assay. The measurement of the MMP-1 level is conducted on the incubation media by means of an ELISA assay.

Fibroblasts [NHDF] are detached from their support by trypsinization. After counting, the cell suspension is sown in 24-well plates in an amount of 1.10⁵ cells/well, in a [D-MEM+10% FCS] medium.

After 24 h of incubation, the culture medium is removed and replaced with fresh medium supplemented with 1% of FCS, without any ascorbic acid and containing the product(s) to be investigated. The cells are replaced in the oven and incubated for 24 h, in an air-CO₂ atmosphere (95%/5%).

After incubation with test solutions, the cells are rinsed with HBSS and then exposed to UV-A (Philips TL-K 40 W ACTINIC BL REFLECTOR tubes) through HBSS. The UV-A irradiation is of about 10 J/cm² for 1 h. After irradiation, the HBSS is replaced with fresh medium containing the assay element.

The cells are replaced at 37° C. and incubated for 24 h. At the end of the assay, the incubation media of the cultures are sampled and frozen at −20° C. while awaiting the assaying of procollagen I and of MMP-1.

In parallel, the cell mats are rinsed with HBSS and treated for an assay of cell proteins (BCA protein assay). A (0.1N) NaOH solution is added into each well. After incubation, the extracts are sampled and transferred into a 96-well plate. 200 μl of BCA reagent are added into each well. After incubation, the absorbance (optical density) is measured at 570 nm with a microplate reader.

The assay of the procollagen I in the samples is carried out with an ELISA assay (Procollagen Type I C-Peptide EIA Kit from R&D Systems® laboratories, France) for detecting and quantifying by fluorescence the C-terminal peptide of human procollagen I.

The MMP-1 activity is measured with an ELISA assay (Human Active MMP-1 from R&D Systems® laboratories, FRANCE) for detecting and quantifying by fluorescence the MMP-1 activity.

In both cases, the quantification of the fluorescence is accomplished in the culture supernatants, according to the instructions provided by the R&D Systems® laboratories, France, the absorbance being measured at 450 nm.

3.2.2) Results.

The assay was carried out on [NHDF] (F06.2H7 strain) fibroblasts cultivated in a [DMEM+1% FCS] medium, in the absence of sodium ascorbate.

The levels of procollagen I and of MMP-1 were measured in the supernatants of the cultures after treatment and irradiation of the cells.

The NHDF cells were treated with the assay complexes CAAL-2, CAAL-3 and CAAL-4, before (24 h) and after (24 h) UVA irradiation. The mixtures were assayed at the concentration indicated in table I above.

Vitamin E (-tocopherol, 1 mM, “VE” hereafter) and Na ascorbate (50 μg/ml) “ASC” hereafter were tested in parallel as positive controls.

The concentrations of procollagen I [PIP] c-terminal peptide and of metalloproteinase-1 [MMP-1] in the different samples (ng/ml) were corrected according to the protein concentrations of the corresponding wells.

The results (average +/− standard deviation, n=3) are collected in table III hereafter.

The dermo-protective potential is determined here by ½ DPC+½ DPM with:

DPC=% of increase in collagen after treatment of the NHDF fibroblast cells with UV in the presence of the complex according to the invention relatively to a control having been subject to a same UV treatment in the absence of the assay complex according to the present invention or other; and

DPM=% of reduction in metalloproteinase-1 after treatment of NHDF fibroblasts with UVs in the presence of the complex according to the invention relatively to a control having been subject to a same UV treatment in the absence of an assay complex according to the present invention or other.

TABLE III

| | | [PIP] | (%) | Stat | [MMP-1] | (%) | Stat |
|---|---|---|---|---|---|---|---|
| (−) | Control 1 | 2.21+/0.011 | 160% | | 0.052+/0.001 | 21% | |
| UV | | | | | | | |
| (+) | Control 2 | 1.38+/0.012 | 100% | | 0.248+/0.002 | 100% | |
| UV | [CAAL-2] | 2.13+/0.011 | 154% | p ≤ 0.01 | 0.201+/0.002 | 81% | p ≤ 0.01 |
| | [CAAL-3] | 2.02+/0.002 | 146% | p ≤ 0.01 | 0.167+/0.001 | 67% | p ≤ 0.01 |
| | [CAAL-4] | 1.70+/0.033 | 123% | p ≤ 0.01 | 0.266+/0.003 | 107% | p ≤ 0.01 |
| | [ASC] | 3.36+/0.016 | 243% | p ≤ 0.01 | -t | | |
| | 50 μg/ml | | | | | | |
| | [VE] 1 mM | — | | | 0.172+/— | 69% | p ≤ 0.01 |

a) Procollagen I.

UVA irradiation (20 J/cm²) induces sharp reduction (−38%=(160−100/160)) of the synthesis of procollagen.

Under the retained experimental conditions, the assay complexes [CAAL-2], [CAAL-3] and [CAAL-4] all induce a significant increase in [PIP], variable depending on the relevant complex with respect to a control not having been subject to the same UV treatment but in the absence of the assay complex according to the present invention or other. The strongest increase is recorded with [CAAL-2] which is capable of totally restoring synthesis of procollagen I, depressed by UVs.

The 3 assayed complexes may be classified according to the dermo-protective potential towards collagen (DPC) in the following way:

[CAAL-2] (DPC=+54%)>[CAAL-3] (DPC=+46%)>>[CAAL-4] (DPC=+23%)

Under the same experimental conditions, sodium ascorbate ([ASC], 50 μg/ml) is capable of restoring neosynthesis of the collagen I depressed by the UVs with a DPC score=143%.

b) MMP-1.

UVA exposure of the NHDF cells is expressed by a very strong increase in MMP-1.

The assay elements [CAAL-2] and [CAAL-3] induce a significant decrease of MMP-1. The maximum effect is recorded after treatment with [CAAL-3] with DPM=33% reduction of MMP-1.

Conversely, the mixture [CAAL-4] does not exhibit any significant dermo-protective effect.

The 3 assayed complexes may be classified according to the dermo-protective potential towards MMP-1, in the following way:

[CAAL-3] (DPM=+34%)>[CAAL-2] (DPM=+19%)>>[CAAL-4] (DPM≈0%)

Under the same experimental conditions, vitamin E (VE, 1 mM) reduces the MMP-1 level induced by the UV with a DPM score=31%.

4) SKIN ANTI-AGEING POTENTIAL (“AAC” hereafter).

The skin anti-ageing potential ([AAC]) was calculated from the experimental data according to:

[A.A.C]=[AOE]+½.[DPC]+½.[DPM]

with:

[AOE]: % of epidermal antioxidant potential as defined above.

[DPC.]: % of dermo-protective Collagen potential as defined above.

[DPM.]: % of dermo-protective MMP1 potential as defined above.

The results of the 3 assayed mixtures are collected below.

CAAL-2: AOE=96%, DPC=54%, DPM=19, AAC=132.5%;

CAAL-3: AOE=73%, DPC=46%, DPM=34%, AAC=113%;

CAAL-4: AOE=39, DPC=23, DPM=0, AAC=50.5;

The positive controls VE and ASC are reference molecules for each compartment: ASC for the epidermal compartment and VE for the dermal compartment. Nevertheless, if their indexes [A.A.C] are calculated, one obtains for VE: [AAC]=96.5 and for ASC [AAC]=71.5. (VE: AOE=81%, DPC=0, DPM=31%, AAC=96.5% and ASC: AOE=0, DPC=143%, DPM=0, AAC=71.5%).

Under the retained experimental conditions, the results of the study indicate that the 3 complexes exhibit a significant anti-ageing potential which clearly varies according to the relevant complex. The results give the possibility of classifying, according to the anti-ageing potential, the 3 complexes in the following way:

[CAAL-2]>[CAAL-3]>>[CAAL-4]

On the basis of the retained biomarkers (ROS, pro-collagen and MMP-1), and taking into account their important role in the skin ageing process, the capability exhibited by the complex [CAAL-2], on the one hand, of modulating the production of the ROS at the epidermis and, of stimulating neosynthesis of the collagen on the other hand and reducing the production of MMPs at the dermis appears as highly favorable and gives the possibility of concluding that the sought anti-ageing activity is the highest.

The mixture [CAAL-2] with an [AAC] of 132.5 has the highest score of the 3 assayed mixtures. The benefit of the complexes CAAL-2 and CALL-3 is the anti-ageing effectiveness at the 2 epidermal and dermal skin compartments on the one hand and as regards the dermis both an increase in the collagen and a reduction in metalloproteinase-1.

### EXAMPLE 2

### Comparative Study of the Anti-Age Potential of the Complex [CAAL-3] as Compared with 3 Binary Combinations of Actives

The 4 following mixture combinations were tested with the ingredients and according to procedure described in Example 1.
M1: complex [CAAL-3]
M2: binary combination [thalassin+prickly pear]
M3: binary combination [thalassin+hyaluronic acid]
M4: binary combination [prickly pear+hyaluronic acid]

The 4 experimental mixtures [M1], [M2], [M3] and [M4] were directly prepared in the culture media, from the 3 actives according to the formulations hereafter:

| | Actives | | | |
|---|---|---|---|---|
| | 15.736 | 15.737 | 15.738 | 15.739 |
| THALASSIN 2 | 1.6% | 1.6% | 1.6% | — |
| PRICKLY PEAR VEGETABLE | 0.16% | 0.16% | — | 0.16% |
| OIL | | | | |
| HYALURONIC ACID | 0.16% | — | 0.16% | 0.16% |

I. ANTIOXIDANT EPIDERMAL POTENTIAL—DCFH assay:

Study carried out on human HaCaT keratinocytes

Assay conditions:
1. Pretreating the cells for 24 h, with the assay elements, before irradiation of the cells,
2. Exposure of the cells to UV-B (20 mJ/cm²), in the absence of the assay elements,
3. Incubation of the irradiated cells for 24 h, in the presence of the assay elements,
4. Quantification of the intracellular oxidation level (ROS) by measuring the fluorescence of DCFH.

Results:

The intracellular oxidation level (RFU/well) recorded at the control and treated cultures were corrected according to the average level of cell proteins (μg/well). The final values ([ROS]) are expressed in arbitrary units:

[ROS]=(RFU/μg prot.) with RFU=Relative Fluorescence Unit.

Batches treated [MIXTURES]: the 4 assay elements were 100% tested in a [DMEM+FCS(1%)] medium.

The results (average +/− standard deviation, n=3) are gathered in the table hereafter.

TABLE IV

| | |
|---|---|
| DCFH | Stat |

(−) UV

| | |
|---|---|
| 11.65 +/− 0.70 | (%) |

(+) UV

| | | | |
|---|---|---|---|
| 152.17 +/− 5.67 | 140.52 | 100% | |
| 52.89 +/− 1.77 | 41.24 | 29% | p ≤ 0.01 |
| 68.51 +/− 1.09 | 56.86 | 40% | p ≤ 0.01 |
| 89.88 +/− 2.05 | 78.23 | 56% | p ≤ 0.01 |
| 106.51 +/− 1.13 | 94.86 | 62% | p ≤ 0.01 |
| 28.03 +/− 1.12 | 16.38 | 12% | p ≤ 0.01 |

Exposure of the HaCaT cells to UVs is expressed by a very clear increase in the intracellular oxidation level. The basal oxidation level (non-irradiated cultures) is multiplied by 13 after UV exposure of the cells.

The results show that the treatment of the cells with the different combinations [M1], [M2], [M3] and [M4] gives the possibility of very clearly reducing the level of intracellular ROSes induced by UVs (ROSUV). It should be noted that the antioxidant epidermal potential (AOE) clearly varies according to the relevant mixture and that the latter is the highest for the ternary mixture:
M1: AOE=71% reduction of [ROS_{UV}] (p≤0.01, Student's test t)
M2: AOE=60% reduction of [ROS_{UV}] (p≤0.01, Student's test t)
M3: AOE=44% reduction of [ROS_{UV}] (p≤0.01, Student's test t)

M4: AOE=38% reduction of [ROS_{UV}] (p≤0.01, Student's test t)

Under the same experimental conditions, the treatment of the cells with vitamin E (α-tocopherol, 1 mM) gives the possibility of reducing by 88% the oxidative stress induced by UVs. This result validates the assay.

II. DERMO-PROTECTIVE POTENTIAL:

Study carried out on human dermal fibroblasts (NHDF)

Assay conditions:
1. Treating the cells, for 24 h, with the assay elements, before irradiation of the cells,
2. Exposure of the cells to UV-As, in the absence of the assay elements,
3. Incubation of the irradiated cells for 24 h, in the presence of the assay elements,
4. Quantification of the levels of pro-collagen I ([PIP] assay) and of MMP-1 in the incubation media.

Results: The [PIP] and [MMP] values recorded at the control and treated cultures were corrected according to the average level of cell proteins (μg/well).

Synthesis of procollagen I: assay of the [PIP] (procollagen of the type I peptide) assay in the incubation media.

positive control: sodium ascorbate (ASC, 50 μg/ml)

MMP-1 production: assay of MMP-1 in the incubation media by an Elisa assay.

positive control: vitamin E (VE, 1 mM)

Treated batches: the 4 assay elements were assayed at 100% in a [DMEM+FCS(1%)] medium.

TABLE V

| | [PIP] | | Stat | [MMP-1] | | Stat |
|---|---|---|---|---|---|---|
| (−) UV | 1.99 +/− 0.02 | 128% | | 0.036 +/− 0.002 | 11% | |
| (+) UV | 1.56 +/− 0.04 | 100% | | 0.317 +/− 0.008 | 100% | |
| | 1.85 +/− 0.03 | 119% | p ≤ 0.01 | 0.189 +/− 0.010 | 60% | p ≤ 0.01 |
| | 1.58 +/− 0.02 | 101% | n.s | 0.184 +/− 0.004 | 58% | p ≤ 0.01 |
| | 1.54 +/− 0.01 | 99% | n.s | 0.210 +/− 0.003 | 66% | p ≤ 0.01 |
| | 1.65 +/− 0.04 | 106% | n.s | 0.175 +/− 0.004 | 55% | p ≤ 0.01 |
| | 3.79 +/− 0.04 | 243% | p ≤ 0.01 | n.t | | |
| | n.t | | | 0.198 +/− 0.006 | 62% | p ≤ 0.01 |

a) Synthesis of collagen I: the UVA irradiation induces a reduction in the synthesis of collagen. Under the retained experimental conditions, only the assay element [M1] induces a significant [PIP] increase relatively to the UV (+) control. The assay element [M1] is capable of partly restoring the neosynthesis of the collagen I depressed by the UVs.

The 4 assayed complexes may be classified, according to the dermo-protective potential towards collagen (DPC), in the following way:

[M1] (DPC=+19%)>>[M2]≈[M3]≈[M4]=(DPC≈0%)

b) Production of MMP-1: UVA exposure of the NHDF cells is expressed by a very strong increase in the production of MMP-1. The assay elements [M1], [M2], [M3] and [M4] induce a significant reduction in MMP-1. It should be noted that the dermo-protective effect (MMP-1) is quasi-similar with the 4 mixtures. The maximum effect is recorded with [M4]: 45% reduction of MMP-1. The 4 assay elements may be classified, according to the dermo-protective potential towards MMP-1 (DPM), in the following way:

[M4] (DPM=45%)≥[M2] (DPM=42%)≈[M1] (DPM=40%)>[M3] (DPM=34%)

Treated batch [ASC]: Under the same experimental conditions, sodium ascorbate (50 μg/ml) is capable of restoring neosynthesis of collagen I depressed by UVs.

Treated batch [VE]: Under the same experimental conditions, vitamin E (1 mM) reduces by 38% (Student's test, p≤0.01) the level of MMP-1 induced by UVs.

CONCLUSION: The skin anti-ageing potential of the 4 experimental mixtures was calculated from experimental data according to:

[A.A.C]=[AOE]+½.[DPC]+½.[DPM]

TABLE VI

| | M1 | M2 | M3 | M4 |
|---|---|---|---|---|
| (AOE) anti-oxidant epidermal potential | 71 | 60 | 44 | 38 |
| (DPC) dermo-protective collagen | 19 | 0 | 0 | 0 |
| potential | | | | |
| (DPM) dermo-protective MMP1 | 45 | 42 | 40 | 34 |
| potential | | | | |
| (AAC) skin anti-aging potential | 103 | 81 | 64 | 55 |

The anti-aging potential of the ternary combination M1 is therefore superior to that of the 3 binary combinations M2, M3 and M4.

EXAMPLE 3

### Cosmetic Compositions for Topical Application

1) CREAM
   A cream was prepared from a mixture of actives CAAL-2 of the following composition:

| TRADE NAME | INCI NAME | SUPPLIER | % by weight |
|---|---|---|---|
| | PURIFIED WATER | | 78.69000 |
| | DISODIUM EDTA | | 0.10000 |
| | GLYCERIN | | 5.00000 |
| SENSIVA ®SC 50 | ETHYLHEXYL GLYCERIN | Seppic (France) | 0.20000 |
| EMULIUM DELTA ® | CETYL ALCOOL and GLYCERYL | Gattefossé | 5.00000 |
| | STEARATE and PEG-75 | (France) | |
| | STEARATE and CETETH-20 and | | |
| | STEARETH-20 | | |
| LANETTE ® 16 | CETYL ALCOHOL | Cognis-BASF | 1.00000 |
| | PRICKLY PEAR OIL | Ies Labo(Fra nce) | 0.16000 |
| MYRITOL ®318 | CAPRYLIC/CAPRIC | Cognis-BASF | 7.00000 |
| | TRIGLYCERIDE | (France) | |
| ARISTOFLEX ®AVC | ACRYLOYLDIMETHYL LAURATE/ | Clariant | 0.50000 |
| | AMMONIUM VP COPOLYMER | (France) | |
| THALASSIN 2 | AQUEOUS SOLUTION OF | BIOTECH | 1.60000 |
| | THALASSIN-2 | MARINE | |
| | | (France) | |
| | PHENOXYETHANOL | | 0.30000 |
| | CHLORPHENESIN | | 0.20000 |
| | LILY HYDROGLYCOLIC | Les Labo | 0.06000 |
| | SOLUTION | (France) | |
| SPIRULIN HS | SPIRULIN HYDROGLYCOLIC | GREENTECH | 0.03000 |
| | SOLUTION | (France) | |
| | SODIUM HYALURONATE | | 0.16000 |
| | 10% SODIUM HYDROXIDE | | Qsp pH 5.67 |
| | | | 100.00000 |

The operating procedure was followed, comprising the following steps:

a) In a mixer, heat to 80° C.:

PURIFIED WATER,

EDTA (preservative), and

CHLORPHENESIN (preservative).

b) Mix and introduce the mixture:

GLYCERIN (moisturizer),

SODIUM HYALURONATE,

c) Disperse with stirring for 30 minutes in a melter into which are introduced and heated to 75° C.:

EMULIUM DELTA® (emulsifier),

LANETTE®16 (emollient), and

MYRITOL® 318 (emollient).

d) Introduce the contents of the melter into the mixer and add:

PRICKLY PEAR OIL, and

ARISTOFLEX® AVC (thickener)

e) Homogenize for 10 minutes and cool to 50° C. and then incorporate:

PHENOXYETHANOL (preservative), and

SENSIVA® SC 50(preservative).

f) Homogenize and introduce:

LILY HYDROGLYCOLIC SOLUTION

SPIRULIN HYDROALCOHOLIC SOLUTION

THALASSIN 2 SOLUTION

g) Homogenize for 10 minutes at 25° C. and adjust the pH to 5.67 by means of a 10% soda (NaOH) solution

2) AQUEOUS GEL

a gel was prepared from the mixture of actives CAAL-2 of the following composition:

| TRADE NAME | INCI NAME | SUPPLIER | % by weight |
|---|---|---|---|
| PURIFIED WATER | | | 91.79000 |
| ULTREZ ®10 | CARBOMER | Lubrizol (France) | 0.20000 |
| GLYCERINE | GLYCERIN | | 5.00000 |
| GEOGARD ®ULTRA | GLUCONOLACTONE, CALCIUM | Lonza (CH) | 0.50000 |
| | GLUCONATE, SODIUM BENZOATE | | |
| JAGUAR ® 105 HP | HYDROXYPROPYL GUAR | Rhodia (France) | 0.30000 |
| TRIETHANOLAMINE | TRIETHANOLAMINE | Interchimie (France) | 0.20000 |
| PRICKLY PEAR OIL | | Les Labo | 0.16000 |
| | | (France) | |
| THALASSIN 2 SOLUTION | | Biotech marine | 1.60000 |
| | | (France) | |
| LILY FLOWER HYDROGLYCOLIC | | Les Labo | 0.06000 |
| SOLUTION | | (France) | |
| SPIRULIN HYDROALCOHOLIC | | Greentech (France) | 0.03000 |
| SOLUTION HS | | | |
| SODIUM HYALURONATE | | Biophil (France) ® | 0.16000 |
| | | | 100.000 |

The gel composition above was prepared by following the operating procedure comprising the following steps.

a) preparation of a phase A

Sprinkle ULTREZ ®10 (gelling agent) in a portion of water and leave at rest for 20 minutes.

b) preparation of a phase B

in a mixer, heat the glycerol (moisturizing agent) with a water portion to 60° C. and dissolve GEOGARD® ULTRA (preservative);

Add SODIUM HYALURONATE and disperse with stirring for 30 minutes, and

At 40° C., add JAGUAR® 105 HP (thickener) and homogenize.

c) Mixing both phases A and B.

Add phase A to phase B with stirring and leave it at rest for 40 minutes,

Neutralize (pH=7) with TRIETHANOLAMINE (pH adjusting agent), and

Introduce: the 4 active ingredients LILY FLOWER SOLUTION, SPIRULIN SOLUTION, THALASSIN 2 SOLUTION and PRICKLY PEAR OIL;

Homogenize for 10 minutes.

1. An anti-age active complex for the skin comprising at least the following elements:
thalassin, extracted from Tripleurospermum Maritimum,
prickly pear seed oil, and
hyaluronic acid or salt thereof.

2. The complex according to claim 1 in liquid form comprising:
0.2 to 3% by weight of an aqueous solution of thalassin with 2% by weight of thalassin,
0.02 to 2% by weight of prickly pear seed oil, and
0.02 to 0.2% by weight of hyaluronic acid or salt thereof.

3. The complex according to claim 1, further comprising at least the following elements:
extract from white lily flower Lilium candidum, and
spirulin, extracted from Spirula Maxima.

4. The complex according to claim 3 in liquid form comprising:
02 to 0.15% by weight of hydroglycolic solution of white lily flower Lilium candidum containing 5% by weight of said white lily flower Lilium candidum extract, and
0.02 to 0.05% by weight of hydroglycolic solution of spirulin containing 1% by weight of spirulin.

5. The complex according to claim 1 in liquid form comprising at least:
1 to 2% by weight of an aqueous solution of thalassin with 2% by weight of thalassin,
0.1 to 0,2% by weight of said prickly pear oil, and
0.1 to 0.2% by weight of hyaluronic acid or salt thereof.

6. The complex according to claim 1 in a liquid solution further comprising at least:
0.01 to 0.1% by weight of a hydroglycolic solution containing 5% by weight of said white lily extract, and
0.01 to 0.1% by weight of a hydroglycolic solution containing 1% by weight of spirulin.

7. The complex according to claim 1 in liquid form comprising:
1.6% by weight of said thalassin solution,
0.16% by weight of said prickly pear oil, and
0.16 mg/ml of hyaluronic acid.

8. The complex according to claim 7 in liquid form comprising:
1.6% by weight of said thalassin solution,
0.16% by weight of prickly pear oil,
0.16% by weight of hyaluronic acid,
0.06% by weight of said hydroglycolic solution of said white lily extract, and
0.03% by weight of said hydroglycolic solution of spirulin.

9. A cosmetic or dermatological product comprising one said complex according to claim 1 in combination with cosmetically or pharmaceutically acceptable supports or excipients.

10. The anti-ageing cosmetic product according to claim 9, characterized in that it is formulated for a topical application as a gel, lotion, cream, pomade, soap, or paste for a mask.
